(19) United States
(12) Patent Application Publication
(10) Pub. No.: US 2002/0052324 A1
(43) Pub. Date: May 2, 2002

(54) Treatment of diabetes with thiazolidinedione, insulin secretagogue and alpha glucocidase inhibitor

(75) Inventors: Robin Edwin Buckingham, Welwyn Garden City (GB); Stephen Alistair Smith, Bramfield (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham plc

(21) Appl. No.: 09/989,572

(22) Filed: Nov. 20, 2001

Related U.S. Application Data
(63) Continuation of application No. 09/445,908, filed on Dec. 15, 1999.
Application No. 09/445,908 is a 371 of international application No. PCT/GB98/02112, filed on Jul. 16, 1998.

(30) Foreign Application Priority Data
Jul. 18, 1997 (GB) GB9715298.7

(51) Int. Cl. A61K 31/70; A61K 31/426

(52) U.S. Cl. 514/23; 514/369

(57) ABSTRACT

A method for the treatment of diabetes mellitus and conditions associated with diabetes mellitus in a mammal, which method comprises administering an effective non-toxic and pharmaceutically acceptable amount of an insulin sensitizer, an insulin secretagogue and an alpha glucosidase inhibitor antihyperglycaemic agent, to a mammal in need thereof; and composition for use in such method.

This invention relates to a method of treatment, in particular to a method for the treatment of diabetes mellitus, especially non-insulin dependent diabetes (NIDDM) or Type 2 diabetes and conditions associated with diabetes mellitus.

Insulin secretagogues are compounds which promote increased secretion of insulin by the pancreatic beta cells.

The sulphonylureas are well known examples of insulin secretagogues. The sulphonylureas act as antihyperglycaemic agents and are used in the treatment of Type 2 diabetes. Examples of sulphonylureas include glibenclamide, glipizide, gliclazide, glimepiride, tolazamide and tolbutamide.

Alpha glucosidase inhibitor antihyperglycaemic agents, such as acarbose, emiglitate and miglitol, are commonly used in the treatment of Type 2 diabetes.

European Patent Application, Publication Number 0,306.228 relates to certain thiazolidinedione derivatives disclosed as having antihyperglycaemic and antihyperlipidaemic activity. One particular thiazolidinedione disclosed in EP 0306228 is 5-[4-[2-(N-methyl-N-(2-pyridyl)amino)ethoxy]benzyl]thiazolidine-2,4-dione (hereinafter ‘Compound (I)’). WO94/05659 discloses certain salts of Compound (I) including the maleate salt.

Compound (I) is an example of a class of anti-hyperglycaemic agents known as ‘insulin sensitisers’. In particular Compound (I) is a thiazolidinedione insulin sensitiser.

European Patent Applications, Publication Numbers: 0008203, 0139421, 0032128, 0428312, 0489663, 0155845, 0257781, 0208420, 0177353, 0319189, 0332331, 0332332, 0528734, 0508740; International Patent Application, Publication Numbers 92/18501, 93/02079, 93/22445 and U.S. Pat. Nos. 5,104,888 and 5,478,852, also disclose certain thiazolidinedione insulin sensitisers.

Another series of compounds generally recognised as having insulin sensitiser activity are those typified by the compounds disclosed in International Patent Applications, Publication Numbers WO93/21166 and WO94/01420. These compounds are herein referred to as ‘acyclic insulin sensitisers’. Other examples of acyclic insulin sensitisers are those disclosed in U.S. Pat. No. 5,232,945 and International Patent Applications, Publication Numbers WO92/03425 and WO91/19702.

Examples of other insulin sensitisers are those disclosed in European Patent Application, Publication Number 0533933, Japanese Patent Application Publication Number 05271204 and U.S. Pat. No. 5,264,451.

The above mentioned publications are incorporated herein by Reference. provides a particularly beneficial effect on glycaemic control, such combination is therefore particularly useful for the treatment of diabetes mellitus. especially Type 2 diabetes, and conditions associated with diabetes mellitus. The treatment is also indicated to proceed with minimum side effects.

Accordingly, the invention provides a method for the treatment of diabetes mellitus, especially Type 2 diabetes and conditions associated with diabetes mellitus, in a mammal such as a human, which method comprises administering an effective non-toxic and pharmaceutically acceptable amount of an insulin sensitiser, an insulin secretagogue and an alpha glucosidase inhibitor antihyperglycaemic agent, to a mammal in need thereof.

The method comprises either co-administration of the insulin sensitiser, an insulin secretasogue and an alpha glucosidase inhibitor antihyperglycaemic agent or sequential administration thereof.

Co-administration includes administration of a formulation which includes an insulin sensitiser, an insulin secretagogue and an alpha glucosidase inhibitor antihyperglycaemic agent or the essentially simultaneous administration of separate formulations of each agent.

In another aspect the invention provides the use of an insulin sensitiser, such as Compound (I), an insulin secretagogue and an alpha glucosidase inhibitor antihyperglycaemic agent, in the manufacture of a composition for the treatment of diabetes mellitus, especially Type 2 diabetes and conditions associated with diabetes mellitus.

A suitable insulin sensitiser is a thiazolidinedione insulin sensitiser.

A suitable thiazolidinedione insulin sensitiser is Compound (I).

Other suitable thiazolidinedione insulin sensitisers include (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone)

Suitable insulin secretagogues include sulphonylureas.

Suitable sulphonylureas include glibenclamide, glipizide, gliclazide, glimepiride, tolazamide and tolbutamide.

Further sulphonylureas include acetohexamide, carbutamide, chlorpropamide, glibomuride, gliquidone, glisentide, glisolamide, glisoxepide. glyclopyamide and glycylamide.

Further suitable insulin secretagogues include repaglinide.

A suitable alpha glucosidase inhibitor antihyperglycaemic agent is acarbose.

Other suitable alpha glucosidase inhibitor antihyperglycaemic agents are emiglitate and miglitol.

In one particular aspect, the method comprises the administration of 2 to 12 mg of Compound (I), especially when administered per day.

Particularly, the method comprises the administration of 2 to 4, 4 to 8 or 8 to 12 mg of Compound (I) per day.

Particularly, the method comprises the administration of 2 to 4 mg of Compound (I), especially when administered per day.

Particularly, the method comprises the administration of 4 to 8 mg of Compound (I), especially when administered per day.

Particularly, the method comprises the administration of 8 to 12 mg of Compound (I), especially when administered per day.

Preferably, the method comprises the administration of 2 mg of Compound (I), especially when administered per day.

Preferably, the method comprises the administration of 4 mg of Compound (I), especially when administered per day.

Preferably, the method comprises the administration of 8 mg of Compound (I), especially when administered per day.

It will be understood that the insulin sensitiser, such as compound (I), the insulin secretagogue and the alpha glucosidase inhibitor antihyperglycaemic agent are each administered in a pharmaceutically acceptable form, including pharmaceutically acceptable derivatives such as pharmaceutically acceptable salts, esters and solvates thereof, as appropriate. In certain instances herein the names used for the relevant insulin secretagogues and the alpha glucosidase inhibitor antihyperglycaemic agents may relate to a particular pharmaceutical form of the relevant active agent: it will be understood that all pharmaceutically acceptable forms of the active agents per se are encompassed by this invention.

Suitable pharmaceutically acceptable salted forms of the insulin sensitisers, such as Compound (I), include those described in the above mentioned patents and patent applications such as in EP 0306228 and WO94/05659 for Compound (I). A preferred pharmaceutically acceptable salt for Compound (I) is a maleate.

Suitable pharmaceutically acceptable solvated forms of the insulin sensitisers. such as Compound (I), include those described in the above mentioned patents and patent applications, such as in EP 0306228 and WO94/05659 for Compound (I), in particular hydrates.

Suitable pharmaceutically acceptable forms of the insulin secretagogue and the alpha glucosidase inhibitor antihyperglycaemic agent depend upon the particular compound used but include known pharmaceutically acceptable forms of the particular compound chosen. Such derivatives are found or are referred to in standard reference texts such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein).

The insulin sensitisers, such as Compound (I) or, a pharmaceutically acceptable salt thereof, or a pharmaceutically acceptable solvate thereof, may be prepared using known methods, for example those disclosed in the above mentioned patents and patent applications, such as EP 0306228 and WO94/05659 for Compound (I). The disclosures of the above mentioned patents and patent applications, such as EP 0306228 and WO94/05659, are incorporated herein by reference.

Compound (I) may exist in one of several tautomeric forms, all of which are encompassed by the term Compound (I) as individual tautomeric forms or as mixtures thereof. Compound (I) contains a chiral carbon atom, and hence can exist in up to two stereoisomeric forms, the term Compound (I) encompasses all of these isomeric forms whether as individual isomers or as mixtures of isomers, including racemates.

The insulin secretagogue and alpha glucosidase inhibitor antihyperglycaemic agent of choice is prepared according to known methods, such methods are found or are referred to in standard reference texts, such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein).

When used herein the term ‘conditions associated with diabetes’ includes conditions associated with diabetes mellitus itself and complications associated with diabetes mellitus. Also included in ‘conditions associated with diabetes’ are those conditions associated with the pre-diabetic state.

When used herein the term ‘conditions associated with the pre-diabetic state’ includes conditions such as insulin resistance, including hereditary insulin resistance, impaired glucose tolerance and hyperinsulinaemia.

‘Conditions associated with diabetes mellitus itself’ include hyperglycaemia, insulin resistance, including acquired insulin resistance. Further conditions associated with diabetes mellitus itself include hypertension and cardiovascular disease, especially atherosclerosis and conditions associated with insulin resistance. Conditions associated with insulin resistance include polycystic ovarian syndrome and steroid induced insulin resistance and gestational diabetes.

‘Complications associated with diabetes mellitus’ includes renal disease, specially renal disease associated with Type 2 diabetes, neuropathy and retinopathy.

Renal diseases associated with Type 2 diabetes include nephropathy, glomerulonephritis, glomerular sclerosis, hypertensive nephrosclerosis and end stage renal disease. Additional renal diseases associated with Type 2 diabetes include nephrotic syndrome.

For the avoidance of doubt, when reference is made herein to scalar amounts, including mg amounts, of Compound (I) in a pharmaceutically acceptable form, the scalar amount referred to is made in respect of Compound (I) per se: For example 2 mg of Compound (I) in the form of the maleate salt is that amount of maleate salt which contains 2 mg of Compound (I).

Diabetes mellitus is preferably Type 2 diabetes.

The particularly beneficial effect on glycaemic control provided by the treatment of the invention is indicated to be a synergistic effect relative to the control expected for the sum of the effects of the individual active agents.

Glycaemic control may be characterised using conventional methods, for example by measurement of a typically used index of glycaemic control such as fasting plasma glucose or glycosylated haemoglobin (HbAlc). Such indices are determined using standard methodology, for example those described in: Tuescher A, Richterich, P., Schweiz. med. Wschr. 101 (1971), 345 and 390 and Frank P., ‘Monitoring the Diabetic Patent with Glycosolated Hemoglobin Measurements’, Clinical Products 1988.

In a preferred aspect, the dosage level of each of the active agents when used in accordance with the treatment of the invention will be less than would have been required from a purely additive effect upon glycaemic control.

Suitably, the insulin sensitiser is the agent of first administration.

Suitably the insulin secretagogue is the agent of second administration.

Suitably the alpha glucosidase inhibitor is the agent of third administration.

There is also an indication that the treatment of the invention will effect an improvement, relative to the individual agents, in the levels of advanced glycosylation end products (AGEs), leptin and serum lipids including total cholesterol, HDL-cholesterol, LDL-cholesterol including improvements in the ratios thereof, in particular an improvement in serum lipids including total cholesterol, HDL-cholesterol, LDL-cholesterol including improvements in the ratios thereof.

As used herein the term ‘pharmaceutically acceptable’ embraces both human and veterinary use: for example the term ‘pharmaceutically acceptable’ embraces a veterinarily acceptable compound.

In the method of the invention, the active medicaments are preferably administered in pharmaceutical composition form. As indicated above, such compositions can include all medicaments or one only of the medicaments.

Accordingly, in one aspect the present invention also provides a pharmaceutical composition comprising an insulin sensitiser, such as Compound (I) and especially 2 to 12 mg thereof, an insulin secretagogue and an alpha glucosidase inhibitor antihyperglycaemic agent and a pharmaceutically acceptable carrier therefor.

Such compositions may be prepared by admixing an insulin sensitiser, such as Compound (I) and especially 2 to 12 mg thereof, an insulin secretagogue and an alpha glucosidase inhibitor antihyperglycaemic agent and a pharmaceutically acceptable carrier therefor.

Usually the compositions are adapted for oral administration. However, they may be adapted for other modes of administration, for example parenteral administration, sublingual or transdermal administration.

The compositions May be in the form of tablets, capsules, powders, granules. lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia gelatin, sorbitol, tragacanth, or polyvinylpyrrolidone: fillers, for example lactose, sugar, maize-starch, calcium phosphate, sorbitol or glycine; tabletting lubricants, for example magnesium stearate: disintegrants, for example starch, polyvinylpyrrolidone, sodium starch glycollate or microcrystalline cellulose; or pharmaceutically acceptable wetting agents such as sodium lauryl sulphate.

The compositions are preferably in a unit dosage form in an amount appropriate for the relevant daily dosage.

Suitable dosages for the insulin sensitisers include those disclosed in the abovementioned patents and patent applications.

Suitable dosages, including unit dosages. of Compound (I) comprise 1, 2, 3, 4, 5, 6, 7, 8, 9, 10, 11 or 12 mg of Compound (I).

In the treatment the medicaments may be administered from 1 to 6 times a day, but most preferably 1 or 2 times per day.

Particular dosages of Compound (I) are 2 mg/day, 4 mg/day, including 2 mg twice per day, and 8 mg/day, including 4 mg twice per day.

Suitable dosages including unit dosages of the insulin secretagogue, such as the sulphonylurea, or the alpha glucosidase inhibitor antihyperglycaemic agent, include the known dosages including unit doses for these compounds as described or referred to in reference text such as the British and US Pharmacopoeias. Remington's Pharmaceutical Sciences (Mack Publishing Co.), Martindale The Extra Pharmacopoeia (London, The Pharmaceutical Press) (for example see the 31st Edition page 341 and pages cited therein).

Thus for the sulphonylureas, a typical daily dosage of glibenclamide is in the range of from 2.5 to 20 mg, for example 10 mg twice per day or 20 mg once per day; a typical daily dosage of glipizide is in the range of from 2.5 to 40 mg: a typical daily dosage of gliclazide is in the range of from 40 to 320 mg; a typical daily dosage of tolazamide is in the range of from 100 to 1000 mg; a typical daily dosage of tolbutamide is in the range of from 1000 to 3000 mg; a typical daily dosage of chlorpropamide is in the range of from 100 to 500 mg; and a typical daily dosage of gliquidone is in the range of from 15 to 180 mg.

Repaglinide may be taken in amounts, usually in the range of from 0.5 mg to 4 mg and usually with meals, up to a typical maximum daily dosage of 16 mg per day.

With regard to the alpha glucosidase inhibitor antihyperglycaemic agents, a typical daily dosage of acarbose is in the range of from 50 to 600 mg, an example 100 mg or 200 mg per day.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations May be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats: emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the Compound (I) is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration.

Composition may, if desired, be in the form of a pack accompanied by written or printed instructions for use.

The compositions are prepared and formulated according to conventional methods, such as those disclosed in standard reference texts, for example the British and US Pharmacopoeias, 's Pharmaceutical Sciences (Mack Publishing Co.), Martindale The Extra Pharmacopoeia (London, The Pharmaceutical Press) and Harry's Cosmeticology (Leonard Hill Books) (for example see the 31st Edition page 341 and pages cited therein).

The present invention also provides a pharmaceutical composition comprising an insulin sensitiser, such as Compound (I) and especially 2 to 12 mg thereof, an insulin secretagogue and an alpha glucosidase inhibitor antihyperglycaemic agent and a pharmaceutically acceptable carrier therefor, for use as an active therapeutic substance.

The invention also provides the use of an insulin sensitiser, such as Compound (I) and especially 2 to 12 mg thereof, an insulin secretagogue and an alpha glucosidase inhibitor antihyperglycaemic agent for the manufacture of a medicament for the treatment of diabetes mellitus and conditions associated with diabetes mellitus.

In particular, the present invention provides a pharmaceutical composition comprising an insulin sensitiser, such as Compound (I) and especially 2 to 12 mg thereof, an insulin secretagogue and an alpha glucosidase inhibitor antihyperglycaemic agent and a pharmaceutically acceptable carrier therefor, for use in the treatment of diabetes mellitus and conditions associated with diabetes mellitus.

A range of 2 to 4 mg includes a range of 2.1 to 4, 2.2 to 4, 2.3 to 4, 2.4 to 4, 2.5 to 4, 2.6 to 4, 2.7 to 4, 2.8 to 4, 2.9 to 4 or 3 to 4 mg.

A range of 4 to 8 mg includes a range of 4.1 to 8, 4.2 to 8, 4.3 to 8, 4.4 to 8, 4.5 to 8, 4.6 to 8, 4.7 to 8, 4.8 to 8, 4.9 to 8, 5 to 8, 6 to 8 or 7 to 8 mg.

A range of 8 to 12 mg includes a range of 8.1 to 12, 8.2 to 12, 8.3 to 12, 8.4 to 12, 8.5 to 12, 8.6 to 12. 8.7 to 12, 8.8 to 12, 8.9 to 12, 9 to 12, 10 to 12 or 11 to 12 mg.

No adverse toxicological effects are expected for the compositions or methods of the invention in the abovementioned dosage ranges.

| Ingredient | Quantity (%) |
|---|---|
| Milled Compound (I) as maleate | 13.25 (pure |
| salt | maleate salt) |
| Sodium Starch Glycollate | 5.00 |
| Hydoxypropyl Methylcellulose | 5.00 |
| 2910 | |
| Microcrystalline Cellulose | 20.0 |
| (Avicel PH102) | |
| Lactose Monohydrate, regular | to 100 |
| grade | |
| Purified water | * |

*Removed during processing.

| | Quantity (mg per Tablet) | | | |
|---|---|---|---|---|
| Tablet Strength | 1.0 mg | 2.0 mg | 4.0 mg | 8.0 mg |
| Active Ingredient: | | | | |
| Compound (I) maleate Concentrate | 10.00 | 20.00 | 40.00 | 80.00 |
| granules | | | | |
| Other Ingredients: | | | | |
| Sodium Starch Glycollate | 6.96 | 6.46 | 5.46 | 10.92 |
| Microcrystalline Cellulose (Avicel | 27.85 | 25.85 | 21.85 | 43.70 |
| PH102) | | | | |
| Lactose monohydrate, (Pharmatose | 104.44 | 96.94 | 81.94 | 163.88 |
| DCL15), | | | | |
| Magnesium Stearate | 0.75 | 0.75 | 0.75 | 1.50 |
| Total Weight of Tablet Core | 150.0 | 150.0 | 150.0 | 300.0 |
| Opadry | 4.5 | 4.5 | 4.5 | 9.0 |
| Total Weight of Film Coated | 154.5 | 154.5 | 154.5 | 309.0 |
| Tablet | | | | |

Compositions of the other active agents are as disclosed in the references mentioned herein.

1. A method for the treatment of diabetes mellitus and conditions associated with diabetes mellitus in a mammal, which method comprises administering an effective non-toxic and pharmaceutically acceptable amount of an insulin sensitiser, an insulin secretagogue and an alpha glucosidase inhibitor antihyperglycaemic agent, to a mammal in need thereof.

2. A method according to claim 1, wherein the insulin secretagogue is glibenclamide, glipizide, gliclazide, glimepiride, tolazamide, tolbutamide, acetohexamide, carbutamide, chlorpropamide, glibomuride, gliquidone, glisentide, glisolamide, glisoxepide, glyclopyamide, glycylamide or repaglinide.

3. A method according to claim 1, wherein the alpha glucosidase inhibitor antihyperglycaemic agent is acarbose, emiglitate and miglitol.

4. A method according to any one of claims 1 to 3, wherein the insulin sensitiser is 5-[4-˜2-(N-methyl-N-(2-pyridyl)amino)ethoxy]benzyl]thiazolidine-2,4-dione (Compound I).

5. A method according to any one of claims 1 to 4, which comprises the administration of 2 to 12 mg of Compound (I).

6. A method according to any one of claims 1 to 5, which comprises the administration of 2 to 4, 4 to 8 or 8 to 12 mg of Compound (I).

7. A method according to any one of claims 1 to 6, which comprises the administration of 2 to 4 mg of Compound (I).

8. A method according to any one of claims 1 to 6, which comprises the method the administration of 4 to 8 mg of Compound (I).

9. A method according to any one of claims 1 to 6, which comprises the administration of 8 to 12 mg of Compound (I).

10. A method according to any one of claims 1 to 6, which comprises the administration of 2 mg of Compound (I).

11. A method according to any one of claims 1 to 6, which comprises the administration of 4 mg of Compound (I).

12. A method according to any one of claims 1 to 6, which comprises the administration of 8 mg of Compound (I).

13. A method according to claim 1, wherein the insulin sensitiser is (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone); or a pharmaceutically acceptable form thereof.

14. A pharmaceutical composition comprising an insulin sensitiser, an insulin secretagogue, an alpha glucosidase inhibitor antihyperglycaemic agent and a pharmaceutically acceptable carrier therefor.

15. A composition according to claim 14, wherein the insulin secretagogue is a sulphonylurea.

16. A composition according to claim 14 or claim 15, wherein the insulin secretagogue is glibenclamide, glipizide, gliclazide, glimepiride, tolazamide or tolbutamide, acetohexamide, carbutamide, chlorpropamide, glibomuride, gliquidone, glisentide, glisolamide, glisoxepide, glyclopyamide, glycylamide or repaglinide.

17. A composition according to claim 1, wherein the alpha glucosidase inhibitor antihyperglycaemic agent is acarbose, emiglitate and miglitol.

18. A composition according to any one of claims 14 to 17, wherein the insulin sensitiser is Compound (I)

19. A composition according to any one of claims 14 to 17, which comprises 2 to 12 mg of Compound (I).

20. A pharmaceutical composition comprising an insulin sensitiser an insulin secretagogue and a pharmaceutically acceptable carrier therefor, for use as an active therapeutic substance.

21. A pharmaceutical composition comprising an insulin sensitiser, an insulin secretagogue, an alpha glucosidase inhibitor antihyperglycaemic agent and a pharmaceutically acceptable carrier therefor, for use in the treatment of diabetes mellitus and conditions associated with diabetes mellitus.

22. A composition according to any one of claim 14, 20 or 21, wherein the insulin sensitiser is (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone); or a pharmaceutically acceptable form thereof.
